(19) United States
(12) Patent Application Publication
(10) Pub. No.: US 2003/0040681 A1
(43) Pub. Date: Feb. 27, 2003

(54) Biopsy apparatus and method of obtaining biopsy sample

(75) Inventors: Raymond H.W. Ng, North York (CA); Victor Daykin, Toronto (CA)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(73) Assignee: Advance Sentry Corporation

(21) Appl. No.: 10/209,668

(22) Filed: Aug. 1, 2002

Related U.S. Application Data
(63) Continuation of application No. 09/522,291, filed on Mar. 9, 2000, now abandoned.
(60) Provisional application No. 60/123,684, filed on Mar. 9, 1999.

(51) Int. Cl. A61B 10/00

(52) U.S. Cl. 600/562

(57) ABSTRACT

A biopsy apparatus is described. The apparatus comprises: a body section having an opening at a distal end thereof; a sample collection means for obtaining a biopsy sample; and a first actuator to provide relative movement between the sample collection means and the body section wherein the sample collection means is operable between a first, retracted position and a second, extended position. A related kit, detachable sample collection device and diagnostic method are also described. The biopsy apparatus is particularly useful as a screen for nasopharyngeal cancer in a patient.

### BACKGROUND OF THE INVENTION

1. Field of the Invention

In one of its aspects, the present invention relates to a biopsy apparatus. In another of its aspects, the present invention relates to a kit for obtaining a biopsy sample. In yet another of its aspects, the present invention relates to a sample collection device for use in a biopsy apparatus. In yet another of its aspects, the present invention relates to a method for diagnosis of nasopharyngeal cancer in a patient.

2. Description of the Prior Art

A biopsy is a procedure whereby a sample of tissue is taken from the body site as a specimen for testing in the laboratory for the purpose of diagnosis or screening of certain medical condition or disease. A biopsy can be performed in a hospital's operating room or in a medical clinic in an ambulatory setting. A Biopsy can range in invasiveness from such procedures as an open lung biopsy for the diagnosis of lung cancer, the closed needle biopsy of internal organ such as examining the kidney for signs of rejection after a kidney transplant, to as routine a procedure as brush biopsy of the cervix for the screening of cervical cancer.

In a cytologic brush biopsy, epithelial cells are collected from the areas of concern by means of brushing or scraping with a bristle brush, a broom brush or a brush specifically designed for that purpose which removes cells from the area being sampled. The collected sample is then placed in a suitable transport medium in a sealed container for shipment to the laboratory where the necessary tests are performed to provide the results for the diagnoses. Epithelial tissues cover all external body surfaces and line all internal spaces and cavities. The skin, mucous membranes, gastrointestinal tract, the lining of the bladder and the lining of the nasopharynx are examples of epithelial tissues. The functions of epithelial tissues are to protect, excrete, and absorb. Cancer originating in the epithelial tissue is called a carcinoma. When localized carcinoma spreads to other parts of the body it metastasizes.

Cytologic brush biopsy is a relatively noninvasive sample collecting procedure as compared to all other biopsy methods and causes minimal pain and discomfort to the patient. Brush biopsy is of particular usefulness where the area from which the samples being collected is easily accessible (such as the skin surface or the mucosal surface); visible or readily identifiable. In most cases, the brush biopsy methodology is for screening purposes, which if results positive shall lead to a diagnostic confirmation by the examination of an additional, larger tissue mass collected by means of an open biopsy.

One of the most commonly performed cytologic brush biopsy is the Papanicolaou smear (“Pap Smear”) for the screening and diagnosis of cervical cancer. The collection steps particular to the brushing action for Pap Smear is generally as follows:

1. Expose the uterine cervix to view and identify the cervical os.

2. Insert the central bristles of the brush into the endocervical canal. Use gentle pressure in the direction of the cervix until the lateral bristles bend against the ectocervix.

3. Maintaining the gentle pressure, rotate the brush four to five times in a clockwise direction, by rolling the shaft between thumb and forefinger.

One particular disease state of which the diagnosis is confirmed by a biopsy procedure is nasopharyngeal carcinoma (NPC). NPC originates in an area of the head deep behind both nasal cavities and above the soft palate, in an area known as the nasopharynx, a portion of the pharynx which lies posterior to the nose and is bounded superiorly by the skull base and sphenoid and laterally by the paired tori of the Eustachian tubes and the Rosenmuller's fossae. Anteriorly the posterior choanae form the limit of the space and inferiorly an artificial line drawn at the level of the hard palate delimits the nasopharynx from the oropharynx.

NPC is rare among North American and European Caucasians with age adjusted incidence rates of less than 1 per 100,000. In contrast, the incidence of NPC in Southern China is in the order of 60 to 80 per 100,000. People who have moved out of the endemic regions maintain their high-risk status and their successive generations also maintain a relatively high-risk status. With an increasing number of Asians immigrating to the US and Canada, NPC has become an important social and medical issue.

At the present time NPC is detected either by visual examination of the nasopharynx or when the patient becomes symptomatic with satellite lesions that are clinically visible. Diagnosis of the lesion is then confirmed by performing a random, mapped or targeted punch biopsy in the nasopharynx after anaesthetizing the patient with a local or general anesthetic. The biopsy samples are then submitted for histologic analysis for the confirmation of diagnosis.

Since NPC originates in an area that is not routinely examined on general physical examination, due to its difficult access and visualization without proper training and availability of special medical instruments such as an endoscope. NPC is often left undetected for a long time without any signs and symptoms until the mass effects of the tumor are apparent. Spread to regional lymph nodes is therefore common and occurs in most patients with NPC. Patients often present with nasal stuffiness, discharge, nose bleeds, ear pain, or decreasing hearing. Cranial nerve involvement is also common and may cause facial muscle signs, swallowing difficulties, facial pain, and aberrant sense of taste. NPC patients with advanced disease have very poor prognosis and survival. Patients with early disease have excellent prognosis and the potential of cure if the treatment is initiated early on. There remains a need for a simple procedure or method that can be performed routinely in an ambulatory setting for mass screening of the presence of NPC, especially early stage disease, in high risk populations.

Epstein Barr virus (“EBV”) is a human DNA tumor virus with an extraordinary diverse oncogenic potential. The association of EBV with NPC was first suggested based on serological evidence. It is well established utilizing Polymerase Chain Reaction (“PCR”) technique that EBV encoded deoxy-ribonucleic acid is present in virtually every biopsied NPC tumor and precancerous epithelial cells, irrespective of histological differentiation. EBV DNA has not been detected in healthy tissues.

EBV is ubiquitous, being found in every population in which it is sought. EBV is carried by 90% of the world adult population. It is exclusively harbored in small subset of B-lymphocytes and is excreted in saliva and in the urogenital tract. Considerable concentrations of infectious virus particles are released at random intervals several times a month. EBV has also been isolated from the cervix and circulates through the blood contained in B-lymphocytes, EBV infections are much more common than the cancers that they produce, infection is usually asymptomatic and malignancy is a relatively rare outcome.

The strength of immune response to EBV has been studied for use as a possible screening tool to predict NPC. The first serological studies demonstrated higher antibody titers and stronger precipitation bands in cases than controls suggesting an etiologic role in the disease. In most Nasopharyngeal carcinoma patients, an elevated serum IgA titer anti-EBV-VCA has frequently been observed, and the degree of elevation is somewhat parallel to the size of NPC tumor mass. Although most Naeopharyngeal tumors will have a positive IgA titer, the majority of positive titers do not reflect a positive tumor. This screening method suffers from unsatisfactory sensitivity or specificity at different cutoff points. The specificity of this test is questionable as only one or two positive test results out of one hundred positive test results will indicate NPC {specificity=(True Negatives)/(True Negative+False Negatives). The specificity of a test screen is an indicator of the ability of the test to classify healthy individuals as having no abnormalities.

While the involvement of EBV in the development of NPC is not clearly understood. It is however, known that EBV infection is a cofactor and a precursor to NPC. EBV typically infects the basal cells, Stratum Basale, of the stratified epithelium through micro lesions of the epithelia. The generation of infected daughter cells begins and continues at the basal layer. These progeny exit the basal layer and by ordinary cell movement migrate to the stratum corneum, which is the uppermost layer of the stratified epithelium. While it may take years for a lesion to be visualized, the EBV infected and cancerous cells are omnipresent in the nasopharynx almost from the first day they begin to migrate from the Stratum Basale. Collection of these cells to screen for the presence of the EBV genome; which is a surrogate marker of malignancy can be performed and the presence of NPC can be predicted. A combination of brush biopsy method for cell sampling and using the sensitive PCR technique for the detection of EBV genome is a superior possibility for a NPC screen, particularly for early disease. PCR instrumentation used to determine the presence of viral genome in the sample sensitive to the number of viral genome copies present at the beginning of the process. The number of EBV genome present at the beginning of the PCR process in part precludes the outcome of the screen.

The nasopharynx is situated deep behind both nasal cavities and samples can be obtained either trans nasally or trans orally. The trans-nasal route is uncomfortable and can be difficult to perform in patients with anatomical abnormalities such as a deviated septum. Furthermore, bleeding can be a problem as the biopsy apparatus transverses the nasal cavities can cause injury to the nasal mucosal surface. The trans oral route is ideal as this is a relatively comfortable and a non-traumatic mean of access to the nasopharynx with minimal or no bleeding. There remains a need for an apparatus to access and perform brush biopsy of the nasopharynx using the trans oral route.

Normally considerable concentrations of infectious virus particles are excreted into saliva at random intervals several times a month and can be residual in the mouth for several days afterwards. Thus, it would be desirable to have a biopsy apparatus designed to obviate the occurrence of contamination with EB virus from saliva or oral mucosal tissue while in transit to the nasopharynx. NPC cells contain substantially greater numbers of vial genome copies than the aggregate of all possible viral contamination from saliva. Thus, it would also be desirous to have a NPC screen with maximum sensitivity by designing the biopsy apparatus such that it minimizes or excludes contact of the sample collection area with saliva or tissue which is not the nasopharynx.

### SUMMARY OF THE INVENTION

It is an object of the present invention to obviate or mitigate at least one of the above-identified disadvantages of the prior art.

It is another object of the present invention to provide a novel biopsy apparatus.

It is yet another object of the present invention to provide a novel kit for obtaining a biopsy sample.

It is yet another object of the present invention to provide a novel sample collection device for use in a biopsy apparatus.

It is yet another object of the present invention to provide a novel method for diagnosis of nasopharyngeal cancer in a patient.

Accordingly, in one of its aspects, the present invention provides A biopsy apparatus comprising:

a body section having an opening at a distal end thereof;

a sample collection means for obtaining a biopsy sample; and

a first actuator to provide relative movement between the sample collection means and the body section wherein the sample collection means is operable between a first, retracted position and a second, extended position.

In another of its aspects, the present invention provides a kit for obtaining a biopsy sample, the kit comprising:

a body section having an opening at a distal end thereof;

a detachable sample collection means for obtaining a biopsy sample; and

a first actuator to provide relative movement between the sample collection means and the body section wherein the sample collection means is operable between a first, retracted position and a second, extended position.

In yet another of its aspects, the present invention provides a detachable sample collection device for use with a biopsy apparatus, the device comprising:

attachment means for removably attaching the sample collection device to the biopsy apparatus, the attachment means comprising an opening; and

a flexible, sample collection surface connected to the attachment means, the flexible, the sample collection surface being moveable through the opening between a first, fold-in position and a second, fold-out position.

In yet another of its aspects, the present invention provides a diagnostic method comprising the steps of:

(i) inserting a distal end of a biopsy apparatus into the mouth of the patient, the apparatus comprising: a body section having an opening at a distal end thereof; a sample collection means for obtaining a biopsy sample; and a first actuator to provide relative movement between the sample collection means and the body section wherein the sample collection means is operable between a first, retracted position and a second, extended position;

(ii) aligning the opening of the biopsy apparatus in the nasopharynx of the patient;

(iii) employing the first actuator to move the sample collection means to the second, extended position;

(iv) brushing the nasopharynx of the patient with the sample collection means to obtain a biopsy sample on the sample collection means;

(v) withdrawing the sample collection means from the nasopharynx of the patient to the first, retracted position;

(vi) withdrawing the biopsy apparatus from the mouth of the patient; and

(vii) assaying the biopsy sample, preferably for the presence of Epstein Barr virus genome in the patient.

As will be apparent to those of skill in the art terms “diagnosis” and “screening” are used interchangeably and are intended to have the same meaning.

Thus, the present inventors have discovered a biopsy device which obviates or mitigates at least one of the foregoing disadvantages of the prior art. The sample collection means of the apparatus is protected by means of a cover or a shroud while the apparatus is in transit through the mouth to the nasopharynx. The cover or shroud is then by exposing the sample collection means immediate to the nasopharynx. After the sample cells are collected, the sample collection means is withdrawn back into the protective cover and then together as part of the device withdrawn from the mouth. Of the patient. Alternatively, the shroud may extend to cover the sample collection means. The cell material is recovered from the brush and transported to the laboratory for analysis.

### BRIEF DESCRIPTION OF THE DRAWINGS

Embodiments of the present invention will be described with reference to the accompanying drawings, in which:

The above as well as other advantages and features of the present invention will be described in greater detail according to a preferred embodiment of the present invention in which:

FIG. 1 is a perspective view of a first embodiment of an apparatus for brush biopsy according to the present invention;

FIG. 2 is a top plan view of the apparatus of FIG. 1;

FIG. 3 is a perspective view of the extended brush of the apparatus of FIG. 1;

FIG. 4 is a side elevation view in section of the brush end of the apparatus of FIG. 1;

FIG. 5 is a side elevation view in section of the apparatus of FIG. 1 in the process of the brush end being extended;

FIG. 6 is a side elevation view in cross section of the apparatus of FIG. 1 with the brush extended;

FIG. 7 is a side elevation view in cross section in view of the apparatus of FIG. 1 and the process of retracting the brush;

FIG. 8 is a side elevation view in cross section of the apparatus of FIG. 1 with the brush fully retracted;

FIG. 9 is a side elevation view illustrating the insertion of the apparatus of FIG. 1 into a mouth;

FIG. 10 is a side elevation view illustrating the elevation of the soft pallet;

FIG. 11 is a side elevation view illustrating the extension of the brush into the nasopharynx cavity;

FIG. 12 is an illustration and side elevation of the brush biopsy sample collection;

FIG. 13 is an illustration and side elevation view of the retraction of the brush;

FIG. 14 is an illustration and side elevation view of the removal of the apparatus from the mouth; and

FIG. 15 is a side elevation view of a second embodiment of the apparatus of the present invention.

### DETAILED DESCRIPTION OF THE PREFERRED EMBODIMENTS

The present invention, in one aspect provides for an apparatus for obtaining cytological samples by brush biopsy. The apparatus allows for the sample to be easily obtained and protected from contamination during the biopsy procedure. A preferred embodiment of the apparatus and its use in obtaining a sample from the nasal pharyngeal is illustrated in the attached figures.

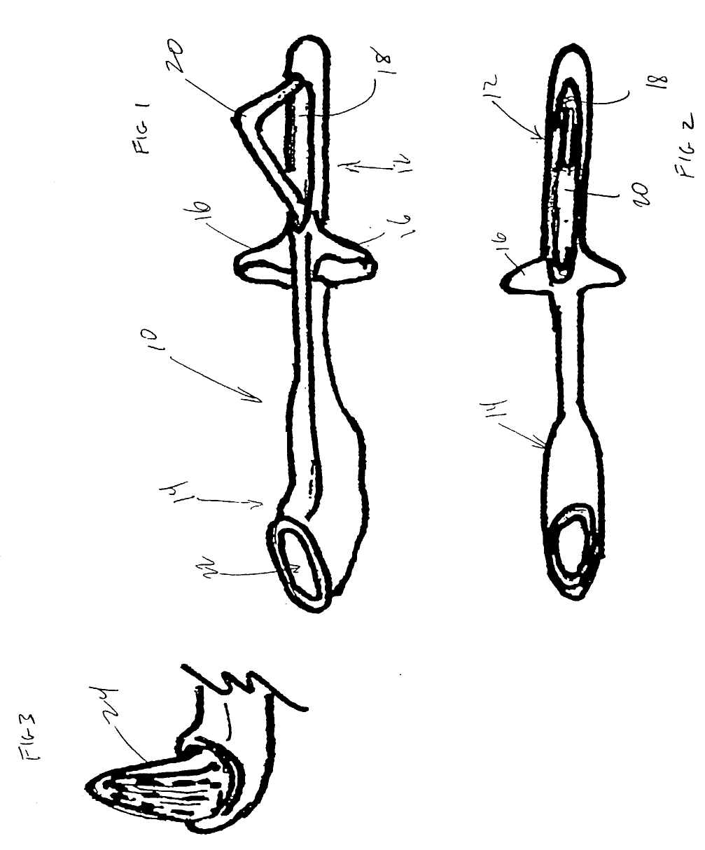

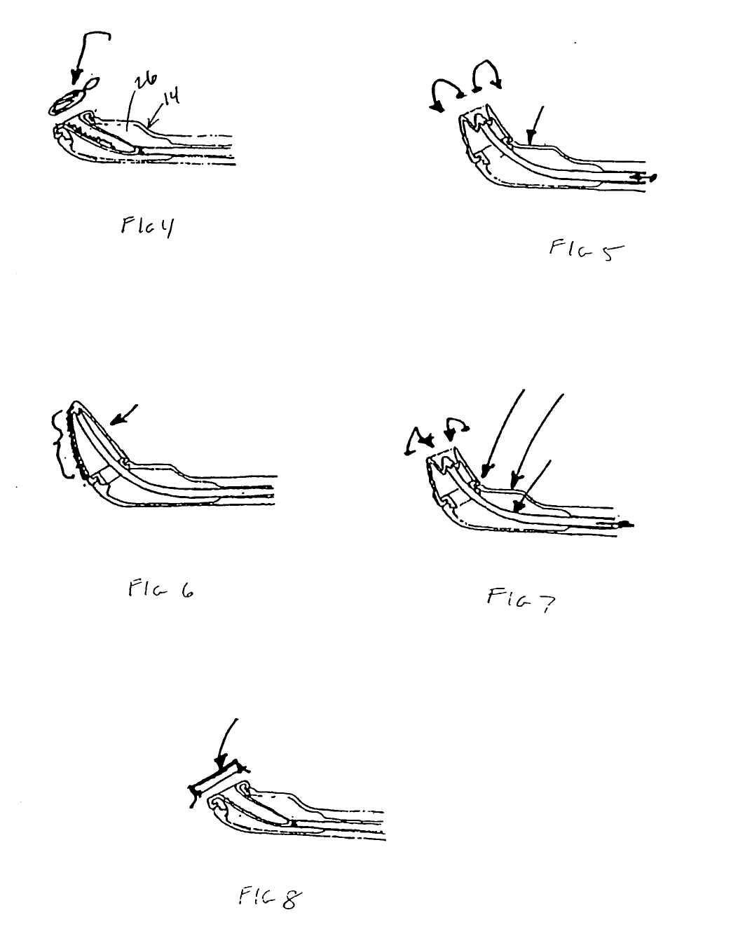

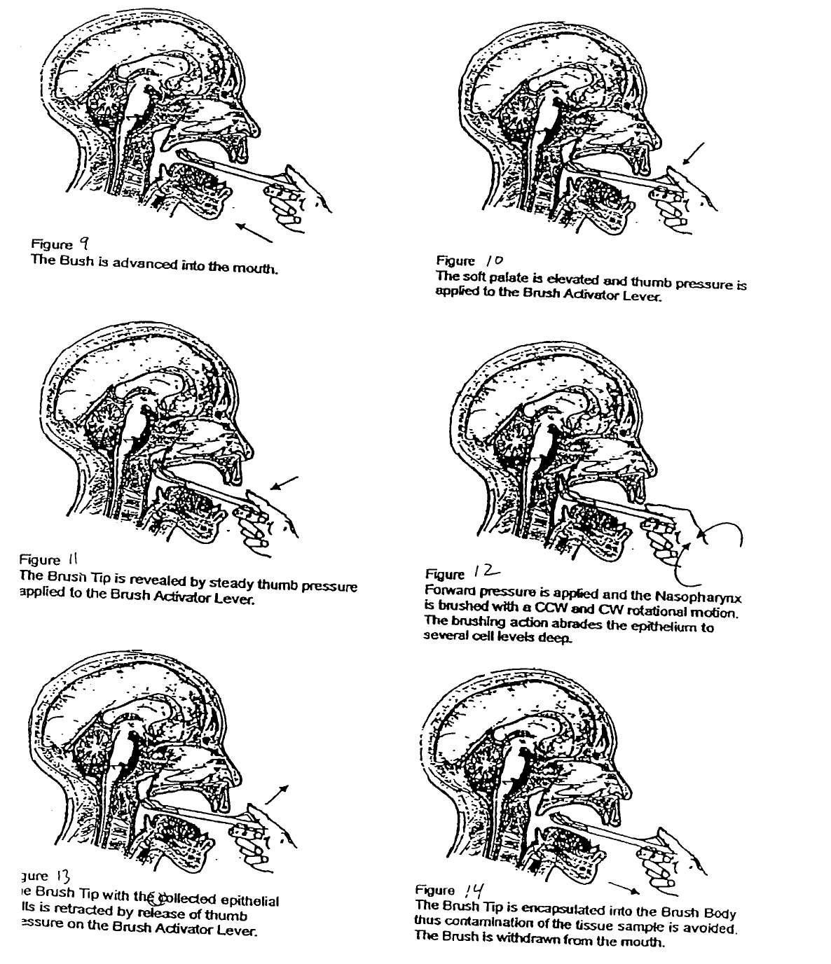

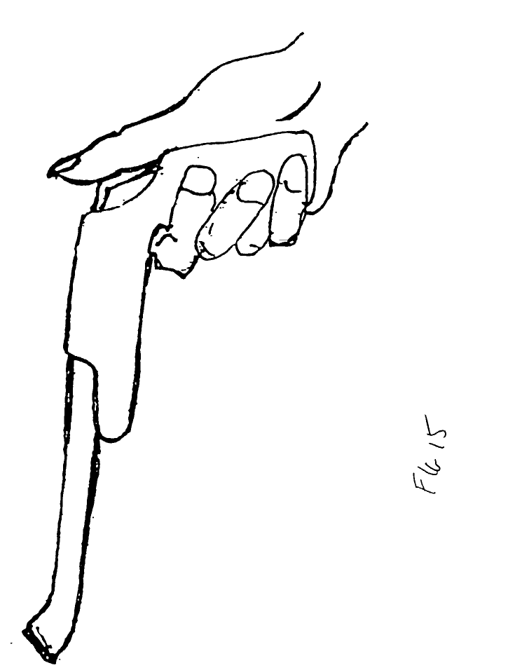

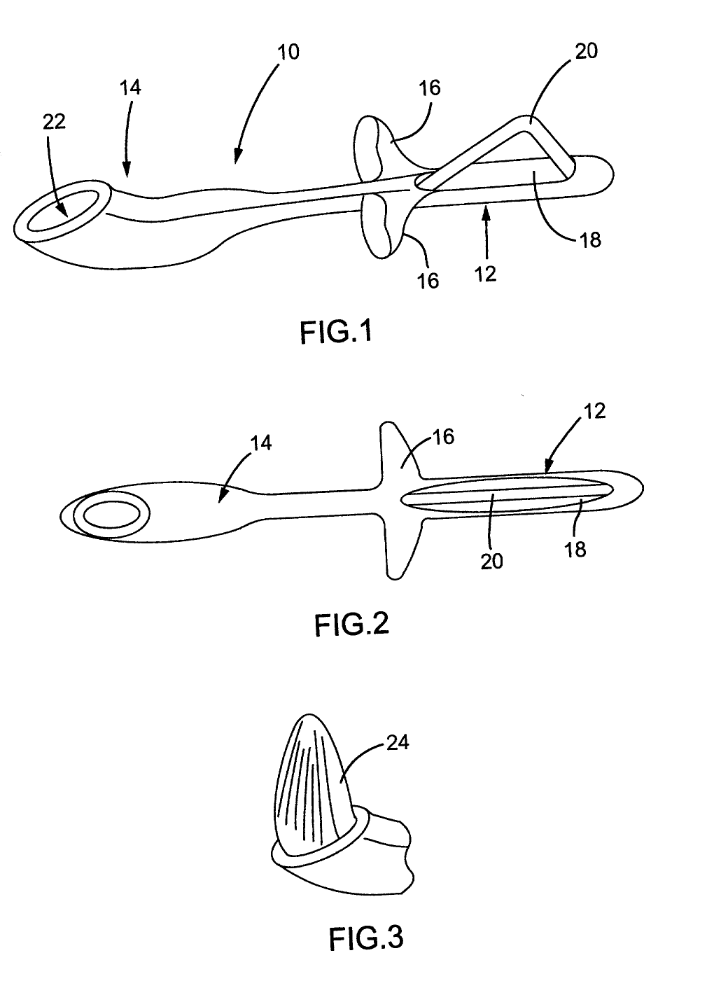

FIG. 1 illustrates a perspective view of a preferred embodiment of the brush biopsy apparatus of the present invention, generally indicated by the numeral 10. The apparatus 10 has a grip region 12 and a sample collection region 14. Grip region 12 is generally a hollow cylinder in shape with finger grips 16 extending on either side to enable the apparatus to be easily held within the hand for manipulation into the proper position for collection of the sample. Apparatus 10 is generally hollow, and the grip region 12 is provided with an opening 18 through which a brush activation lever 20 extends. The operation of the brush activation lever 20 will be described further herein below.

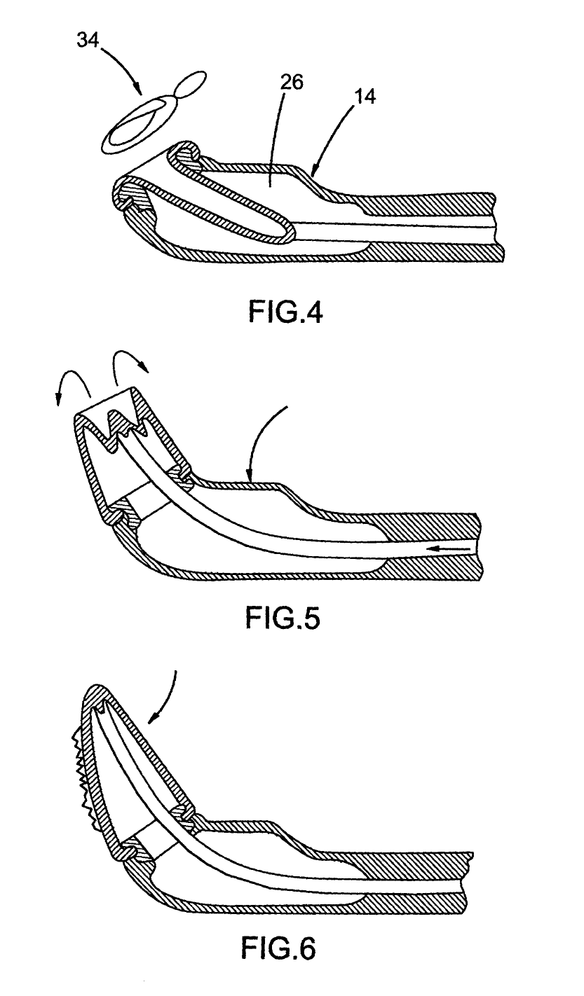

Sample collection region 14 at the end is provided with an aperture 22 through which the sample collection brush 24 is able to extend and retract. FIG. 4 illustrates the sample collection brush 24 in its initial retracted position. Sample collection brush 24 is constructed of a suitable elastomeric material to enable the brush 24 to roll inside out during the operation of the apparatus 10. This elastomeric material may be an evaprene, silicone, rubber, or other suitable biocompatible elastomeric material that will enable the proper operation of the apparatus.

As illustrated in the Figures, the sample collection region 14 is provided with an interior cavity 26 which holds the sample collection brush 24. Sample collection brush 24 has a generally cross-section elliptical shape with the tip 28 of the cone being connected to the end of the brush activation lever 20. The base of the sample collection brush 24 is provided with a rib 30 which is retained within a groove 32 located on the exterior of the aperture 22 of the sample collection region 14. The apparatus 10 is supplied in a sterile condition with the sample collection brush 24 being covered by an aluminum foil peel-off tab 34.

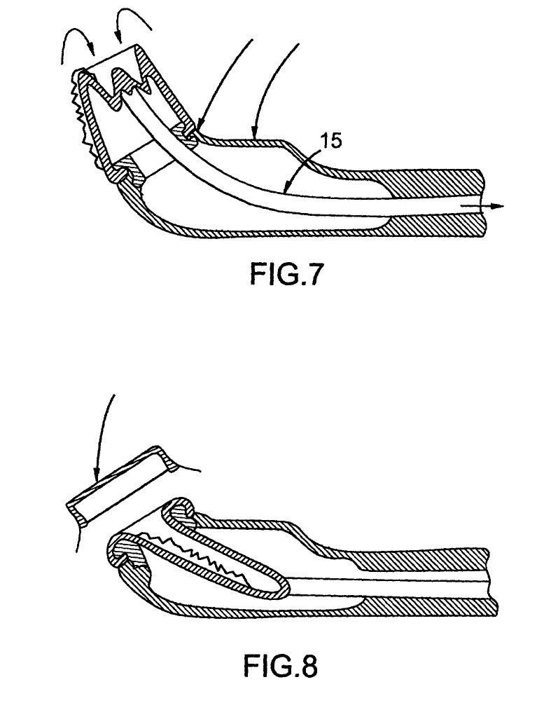

FIGS. 5 through 8 illustrate the extension and retraction of the sample collection brush 24. In FIG. 5, as thumb pressure is placed on the brush activation level 20, the end of the brush activation lever 20 is extended, pushing against the sample collection brush 24. This causes the sample collection brush 24. This causes the sample collection brush 24 to roll inside-out, extending it beyond the end of the aperture 22 of the sample collection region 14. FIG. 6 illustrates the sample collection brush 24 in its fully extended position for the sample collection. Sample collection brush 24 is provided with an abrasive cell collector region 36 which may be a Kraton® (Shell Corporation) or non allergenic thermoplastic elastomers/thermoset rubbers (e.g., silicone rubber) which will provide for the abrasive action of the brush 24 when the sample is collected. Once the sample has been collected, then the brush 24 with the sample attached is retracted into the cavity 26 by releasing the thumb pressure on the brush activation lever 20. This rolls the sample collection brush 24 back into the cavity 26 and protects the collected sample from contamination while it is being withdrawn from the patient. Once the apparatus is withdrawn from the patient, then a suitable transport medium is placed within the sample collection brush 24 and the aperture 22 of the sample collection region 14 covered for transport to a lab. Alternatively, the collected sample may be washed into a conventional transport tube for transport to the laboratory.

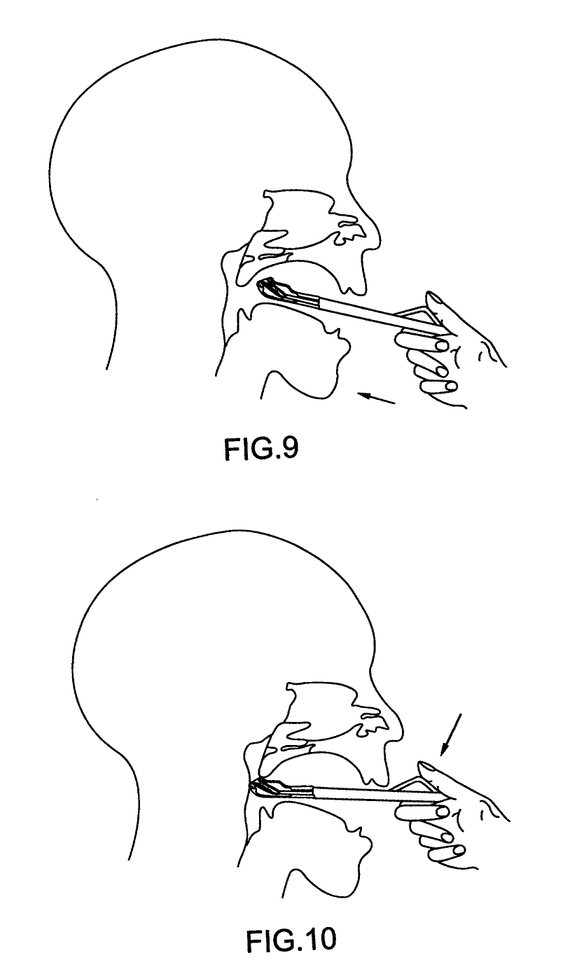

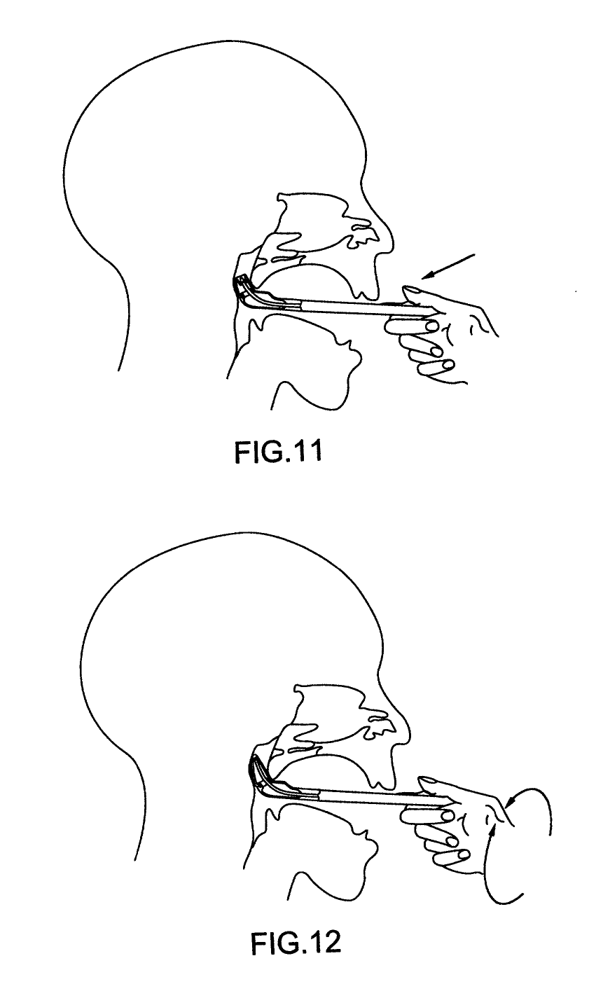

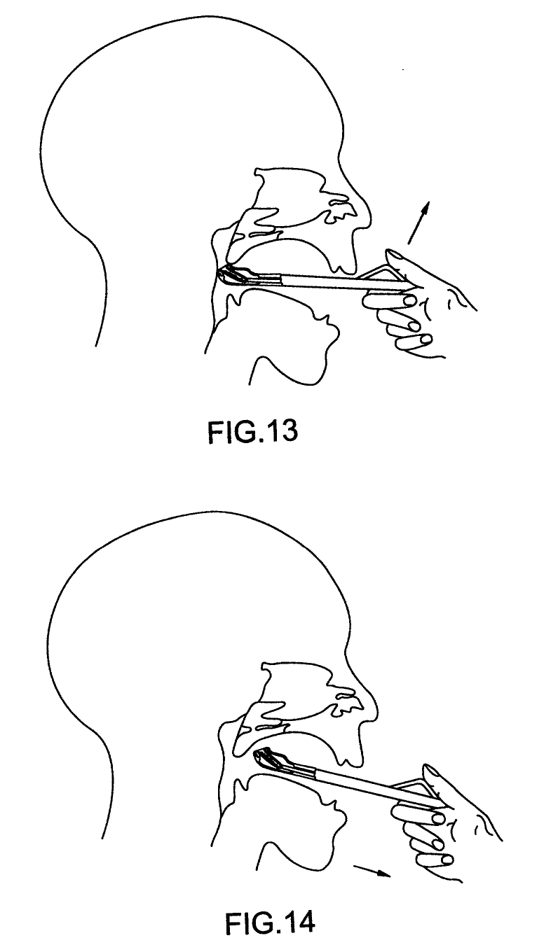

The operation of the apparatus of the present invention for collection of a brush biopsy sample from the nasal pharyngeal by a transoral technique is illustrated in FIGS. 9 to 14. This sample is collected for determination of the presence of nasal pharyngeal carcinoma through a polymerase chain reaction screening for the presence of Epstein Barr virus (EBV) genome.

As illustrated in the Figures, the brush after removal of the foil cover is advanced into the mouth and the soft palate elevated to align the aperture with the nasopharynx of the nasal cavity. Thumb pressure is applied to the brush activator lever to extend the sample collection brush and bring it into contact with the nasopharynx. Once the sample collection brush has been extended, forward pressure is applied and the nasopharynx is brushed with a counterclockwise and clockwise rotational motion (e.g., +20° to −20°_his brushing action abrades the epithelium to several cell levels deep and results in the abraded cell being attached to the cell collection region. Once the brushing action has been completed, the brush tip with the collected epithelial cells is retracted by release of the thumb pressure on the brush activation lever. This results in the sample collection brush being encapsulated back into the cavity 26 of the sample collection zone of the brush body, avoiding contamination of the tissue sample. The brush is then withdrawn from the mouth and the brush may be capped, or the sample may be washed into a suitable transport tube for transport to the laboratory.

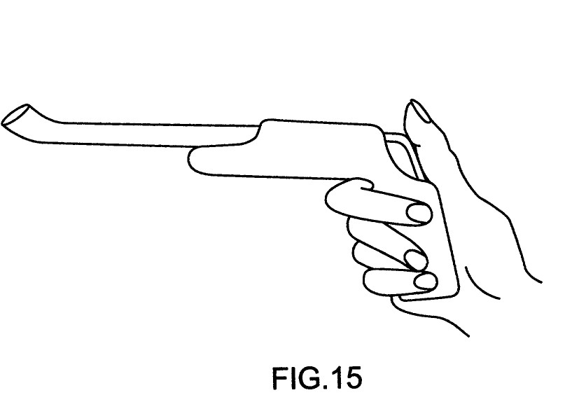

A variation of a sample collection brush, according to the present invention is illustrated in FIG. 15. This variation of the sample collection brush is provided with a pistol grip shape for the grip region. The pistol grip is comfortably held within the hand and the brush activation lever is easily accessible to the thumb for activation of the sample collection brush.

The brush biopsy apparatus of the present invention allows for easy and accurate collection of cytological samples by a brush biopsy technique. The grip region is comfortably held within the hand and allows for very accurate manipulation of the brush apparatus to place the sample collection brush at the appropriate location for the collection of the sample. Once the apparatus is placed at the proper location, the actual sample collection brush is extended from the body of the apparatus to collect the sample by braiding the cells from the area of interest. Once the sample is collected, the sample collection brush is retracted back into the body of the apparatus to protect the sample from contamination, should the apparatus accidentally come into contact with other regions of the body during its insertion or removal from the patient.

The apparatus of the present invention is useful in any brush biopsy procedure for collection of samples by abrasion of the tissue surface. Examples of such procedures include collection of cervical cell samples for PAP smear and other sample collections. The apparatus of the present invention is particularly useful for collection of samples from the nasal pharynx for determination of the presence of Epstein Barr virus genome in the cell samples as an indication of the possible presence of nasopharyngeal carcinoma. The apparatus of the present invention permits the sample to be easily obtained via transoral technique which causes less discomfort to the patient than a trans-nasal brush biopsy, or a needle or punch biopsy. In addition, the design of the apparatus of the present invention allows for the collection of the samples only from the area of interest.

While the invention has been described hereinabove with reference to various preferred embodiments and specific illustrate embodiments, it will be clearly understood by those of skill in the art that modifications to and variations of the preferred embodiments and specific illustrated embodiments are possible which do not depart from the spirit and scope of the present invention. Accordingly, it is contemplated that such modifications to and variations of the preferred embodiments and specific illustrated embodiments are encompassed by the invention.

What is claimed is:

1. A biopsy apparatus comprising:
a body section having an opening at a distal end thereof,
a sample collection means for obtaining a biopsy sample; and
a first actuator to provide relative movement between the sample collection means and the body section wherein the sample collection means is operable between a first, retracted position and a second, extended position.

2. The biopsy apparatus defined in claim 1, wherein, in the first, retracted position, the sample collection means is disposed in a receptacle in the body section.

3. The biopsy apparatus defined in claim 1, wherein, in the second, extended position, the sample collection means is exposed to enable collection of the biopsy sample.

4. The biopsy apparatus defined in claim 1, further comprising a sheath which is retractable with respect to the sample collection means.

5. The biopsy apparatus defined in claim 1, further comprising a sheath which is reversibly retractable with respect to the sample collection means.

6. The biopsy apparatus defined in claim 1, wherein the body section further comprises a resealable cover over the opening.

7. The biopsy apparatus defined in claim 6, wherein the resealable cover is operable between: (i) a first, unsealed position in which the sample collection means may moved therethrough between the first, retracted position and the second, extended position, and (ii) a second, sealed position in which the sample collection means is substantially sealed from exposure to an environment outside the apparatus.

8. The biopsy apparatus defined in claim 1, wherein the body section comprises a first section and a second section angularly disposed with respect to one another.

9. The biopsy apparatus defined in claim 8, wherein the first second and the second section are at an obtuse angle with respect to one another.

10. The biopsy apparatus defined in claim 8, wherein the first second and the second section are at angle in the range of from about 90° to about 135° with respect to one another.

11. The biopsy apparatus defined in claim 8, wherein the first second and the second section are at angle in the range of from about 95° to about 130° with respect to one another.

12. The biopsy apparatus defined in claim 8, wherein the first second and the second section are at angle in the range of from about 100° to about 125° with respect to one another.

13. The biopsy apparatus defined in claim 8, wherein the first second and the second section are at angle in the range of from about 100° to about 115° with respect to one another.

14. The biopsy apparatus defined in claim 8, further comprising angle adjustment means to allow a user to adjust the angle between the first section and the second section.

15. The biopsy apparatus defined in claim 1, wherein the sample collection means comprises a roughened surface.

16. The biopsy apparatus defined in claim 1, wherein the sample collection means is detachable from the apparatus.

17. The biopsy apparatus defined in claim 1, wherein the sample collection means is made of a flexible material.

18. The biopsy apparatus defined in claim 17, wherein, in the first, retracted position, the sample collection means is in a folded-in position and in the second, extended position, the sample collection means is in a folded-out position.

19. The biopsy apparatus defined in claim 1, wherein a slidable shaft interconnects the sample collection means and the first actuator.

20. The biopsy apparatus defined in claim 19, wherein the slidable shaft detachably interconnects the sample collection means and the first actuator.

21. The biopsy apparatus defined in claim 1, further comprising a second actuator to provide movement of the sample collection means in the second, extended position.

22. The biopsy apparatus defined in claim 21, wherein the second actuator provides rotational movement of the sample collection means in the second, extended position.

23. The biopsy apparatus defined in claim 21, wherein the body section comprises a moveable window over the opening.

24. The biopsy apparatus defined in claim 21, wherein the body section comprises a moveable window over the opening, the moveable window connected to the first actuator.

25. The biopsy apparatus defined in claim 1, further comprising a removable cover for connection to the sample collection means after collection of the biopsy sample.

26. A kit for obtaining a biopsy sample, the kit comprising:
a body section having an opening at a distal end thereof;
a detachable sample collection means for obtaining a biopsy sample; and
a first actuator to provide relative movement between the sample collection means and the body section wherein the sample collection means is operable between a first, retracted position and a second, extended position.

27. The kit defined in claim 26, wherein, in the first, retracted position, the sample collection means is disposed in a receptacle in the body section.

28. The kit defined in claim 26, wherein, in the second, extended position, the sample collection means is exposed to enable collection of the biopsy sample.

29. The kit defined in claim 26, further comprising a sheath which is retractable with respect to the sample collection means.

30. The kit defined in claim 26, further comprising a sheath which is reversibly retractable with respect to the sample collection means.

31. The kit defined in claim 26, wherein the body section further comprises a resealable cover over the opening.

32. The kit defined in claim 31, wherein the resealable cover is operable between: (i) a first, unsealed position in which the sample collection means may moved therethrough between the first, retracted position and the second, extended position, and (ii) a second, sealed position in which the sample collection means is substantially sealed from exposure to an environment outside the apparatus.

33. The kit defined in claim 26, wherein the body section comprises a first section and a second section angularly disposed with respect to one another.

34. The kit defined in claim 33, wherein the first second and the second section are at an obtuse angle with respect to one another.

35. The kit defined in claim 33, wherein the first second and the second section are at angle in the range of from about 90° to about 135° with respect to one another.

36. The kit defined in claim 33, wherein the first second and the second section are at angle in the range of from about 95° to about 130° with respect to one another.

37. The kit defined in claim 33, wherein the first second and the second section are at angle in the range of from about 100° to about 125° with respect to one another.

38. The kit defined in claim 33, wherein the first second and the second section are at angle in the range of from about 100° to about 115° with respect to one another.

39. The kit defined in claim 26, further comprising angle adjustment means to allow a user to adjust the angle between the first section and the second section.

40. The kit defined in claim 26, wherein the sample collection means comprises a roughened surface.

41. The kit defined in claim 26, wherein the sample collection means is made of a flexible material.

42. The kit defined in claim 41, wherein, in the first, retracted position, the sample collection means is in a folded-in position and in the second, extended position, the sample collection means is in a folded-out position.

43. The kit defined in claim 26, wherein a slidable shaft interconnects the sample collection means and the first actuator.

44. The kit defined in claim 43, wherein the slidable shaft detachably interconnects the sample collection means and the first actuator.

45. The kit defined in claim 26, further comprising a second actuator to provide movement of the sample collection means in the second, extended position.

46. The kit defined in claim 45, wherein the second actuator provides rotational movement of the sample collection means in the second, extended position.

47. The kit defined in claim 45, wherein the body section comprises a moveable window over the opening.

48. The kit defined in claim 45, wherein the body section comprises a moveable window over the opening, the moveable window connected to the first actuator.

49. The kit defined in claim 26, further comprising a removable cover for connection to the sample collection means after collection of the biopsy sample.

50. A detachable sample collection device for use with a biopsy apparatus, the device comprising:
attachment means for removably attaching the sample collection device to the biopsy apparatus, the attachment means comprising an opening; and
a flexible, sample collection surface connected to the attachment means, the flexible, the sample collection surface being moveable through the opening between a first, fold-in position and a second, fold-out position.

51. The device defined in claim 50, wherein the sample collection surface comprises a roughened surface.

52. The device defined in claim 50, wherein the attachment means provides for mechanical attachment of the device to the biopsy apparatus.

53. The device defined in claim 52, wherein the attachment means includes a threaded section for threaded engagement between the device the biopsy apparatus.

54. The device defined in claim 50, further comprising a removable cover for connection to the sample collection means after collection of the biopsy sample.

55. A diagnostic method comprising the steps of:
(i) inserting a distal end of a biopsy apparatus into the mouth of the patient, the apparatus comprising: a body section having an opening at a distal end thereof; a sample collection means for obtaining a biopsy sample; and a first actuator to provide relative movement between the sample collection means and the body section wherein the sample collection means is operable between a first, retracted position and a second, extended position;
(ii) aligning the opening of the biopsy apparatus in the nasopharynx of the patient;
(iii) employing the first actuator to move the sample collection means to the second, extended position;
(iv) brushing the nasopharynx of the patient with the sample collection means to obtain a biopsy sample on the sample collection means;
(v) withdrawing the sample collection means from the nasopharynx of the patient to the first, retracted position;
(vi) withdrawing the biopsy apparatus from the mouth of the patient; and
(vii) assaying the biopsy sample.

56. The diagnostic method defined in claim 55, comprising the further step, after Step (i), of lifting the soft palate of the patient.

57. The diagnostic method defined in claim 55, wherein, in the first, retracted position, the sample collection means is disposed in a receptacle in the body section.

58. The diagnostic method defined in claim 55, wherein, in the second, extended position, the sample collection means is exposed to enable collection of the biopsy sample.

59. The diagnostic method defined in claim 55, wherein the body section further comprises a resealable cover over the opening.

60. The diagnostic method defined in claim 55, wherein the body section of the biopsy apparatus comprises a first section and a second section angularly disposed with respect to one another.

61. The diagnostic method defined in claim 60, wherein the first second and the second section are at an obtuse angle with respect to one another.

62. The diagnostic method defined in claim 60, wherein the first second and the second section are at angle in the range of from about 90° to about 135° with respect to one another.

63. The diagnostic method defined in claim 60, wherein the first second and the second section are at angle in the range of from about 95° to about 130° with respect to one another.

64. The diagnostic method defined in claim 60, wherein the first second and the second section are at angle in the range of from about 100° to about 125° with respect to one another.

65. The diagnostic method defined in claim 60, wherein the first second and the second section are at angle in the range of from about 100° to about 115° with respect to one another.

66. The diagnostic method defined in claim 60, wherein the biopsy apparatus angle adjustment means to allow a user to adjust the angle between the first section and the second section.

67. The diagnostic method defined in claim 55, wherein the sample collection means comprises a roughened surface.

68. The diagnostic method defined in claim 55, comprising the further step, after Step (vi), of detaching the sample collection means from the biopsy apparatus.

69. The diagnostic method defined in claim 55, wherein the sample collection means is made of a flexible material.

70. The diagnostic method defined in claim 55, wherein Step (iii) comprises moving the sample collection means from a first, retracted folded-in position to a second, extended fold-out position.

71. The diagnostic method defined in claim 55, wherein a slidable shaft interconnects the sample collection means and the first actuator.

72. The diagnostic method defined in claim 71, wherein the slidable shaft detachably interconnects the sample collection means and the first actuator.

73. The diagnostic method defined in claim 55, wherein Step (iv) comprises moving the biopsy apparatus with the sample collection means in the second, extended position.

74. The diagnostic method defined in claim 55, wherein Step (iv) comprises rotating the biopsy apparatus with the sample collection means in the second, extended position.

75. The diagnostic method defined in claim 55, wherein Step (vii) comprises assaying the biopsy sample for the presence the Epstein-Barr viral genome.

76. The diagnostic method defined in claim 55, wherein Step (v) is conducted prior to Step (vi).
